(19) United States
(12) Patent Application Publication
(10) Pub. No.: US 2011/0295115 A1
(43) Pub. Date: Dec. 1, 2011

(54) Interventions Using Correlated Nuclear and Ultrasound Imaging

(76) Inventor: Stephen T. Yarnall, Poway, CA (US)

(21) Appl. No.: 13/116,644

(22) Filed: May 26, 2011

Related U.S. Application Data
(60) Provisional application No. 61/348,730, filed on May 26, 2010.

(51) Int. Cl. A61B 6/00; A61B 8/00

(52) U.S. Cl. 600/436

(57) ABSTRACT

An apparatus and method for localizing a nuclear-emitting lesion during an intervention using correlated nuclear and ultrasound imaging. The apparatus provides interventional access and quasi-stereotactic positioning of interventional devices, with real-time ultrasound image visualization for tracking the approach of the device to the lesion. The apparatus is intended to overcome the shortcomings of fully-stereotactic nuclear-emission image localization.

### CROSS-REFERENCE TO RELATED APPLICATION

The present application claims the benefit under 35 U.S.C. §119(e) of U.S. Provisional Application No. 61/348,730, filed May 26, 2010, which is incorporated herein by reference.

### FIELD OF THE INVENTION

The present invention relates generally to the field of medical imaging. More specifically, the invention relates to systems and methods for using nuclear-emission imaging to localize lesions for tissue harvesting or marking.

### BACKGROUND OF THE INVENTION

In medicine, nuclear-emission imaging is commonly utilized to identify regions (lesions) that may contain abnormal cells, and must be sampled, marked or otherwise treated using interventional devices. Stereotactic methods using nuclear-emission imaging have been developed to position a device in relation to a lesion for performing an intervention; however, guiding interventions using these methods can be problematic due to procedure time constraints, lesion movement, interventional device control, and the three-dimensional resolving power of the nuclear image.

Methods of guiding interventions using nuclear emission-imaging and nuclear-emission imaging in conjunction with anatomic imaging have been disclosed in literature. In comparison with guiding interventions using correlated nuclear emission and ultrasound imaging as with the present invention, existing methods lack clinically important characteristics including: a) clinical expediency, b) the ability to accommodate the necessary redirection of the interventional device in real time during insertion due to tissue characteristics, lesion movement, or mis-targeting, c) the ability to verify the location and progress of the intervention, d) freedom from the use of additional doses of ionizing radiation for extra nuclear imaging procedures, and e) freedom from reliance on cumbersome radioactive fiducial instruments. Examples of these methods include:

One example of an existing method is disclosed in U.S. Pat. No. 4,977,505, Means to correlate images from scans taken at different times including means to determine the minimum distances between a patient anatomical contour and a correlating surface, issued to Pelizzari, et al. Pelizzari discloses correlative methods to improve precision of applied clinical techniques (such as surgery) to nuclear emission identified regions of interest. However, Pelizzari fails to disclose an apparatus or method means to utilize precise correlative methods without additional ionizing radiation.

U.S. Pat. No. 5,961,457, Method and apparatus for radiopharmaceutical-guided biopsy, issued to Raylman, et al. discloses a method of localization using nuclear emission in three dimensions. Raylman also describes a method of utilizing radioactive fiducial marker to confirm localization. However, Raylman fails to teach apparatus or method to perform the procedure within clinically-acceptable time limits, to accommodate necessary redirection of interventional device during insertion due to tissue characteristics or mis-targeting, and to confirm the actual location of the interventional device with respect to the lesion. It only confirms the location of a surrogate fiducial ‘obturator’.

U.S. Pat. No. 6,055,450, Bifurcated gamma camera system, issued to Ashburn discloses means for guiding an intervention based exclusively on nuclear-emission imaging. However, Ashburn fails to teach an apparatus or method to perform the procedure within clinically-acceptable time limits, to accommodate necessary redirection of interventional device during insertion due to tissue characteristics or mis-targeting, or to precisely define the location of the lesion in three dimensions. Ashburn also requires the use of a radioactive fiducial.

US patent application 2003/0153830, Open-access emission tomography scanner by Weinberg, et al. discloses means for guiding an intervention based on nuclear-emission imaging. Weinberg discloses means for stabilizing tissue for generally correlative nuclear-emission and x-ray imaging, and means to provide access to tissue held stable with respect to space coordinates of correlated images. However, Weinberg fails to disclose an apparatus or method for accommodating necessary redirection of interventional device during insertion due to tissue characteristics or mis-targeting, and to confirm the actual location of the interventional device with respect to the emission image.

Patent application PCT/US2006/023940, Tissue interventions using nuclear emission guidance by Yarnall, et al. discloses a means for guiding an intervention based exclusively on nuclear-emission imaging and means to provide access to tissue with respect to space coordinates of emission images. However, Yarnall fails to disclose an apparatus or method to perform the procedure within clinically-acceptable time limits, to accommodate necessary redirection of the interventional device during insertion due to tissue characteristics or mis-targeting, or to precisely define the location of the lesion in three dimensions. Yarnall also requires the use of a radioactive fiducial.

US patent application 2010/0016865, Gamma guided stereotactic localization system by Kieper, et al. discloses a method of using a slant-hole collimator for determining the inter-detector position of a gamma-emitting region of interest. However, Kieper fails to a to disclose an apparatus or method to perform the procedure within clinically-acceptable time limits, to accommodate necessary redirection of interventional device during insertion due to tissue characteristics or mis-targeting, or to precisely define the location of the lesion in three dimensions. Kieper also requires the use of a radioactive fiducial.

### SUMMARY OF THE INVENTION

The present invention is intended to simplify nuclear-emission guided tissue interventions. Of particular interest is simplifying interventions of breast lesions identified using nuclear-emission imaging by using correlated ultrasound imaging for real-time targeting, for guiding the trajectory of interventional devices and for verifying the success of the intervention.

In one aspect of the present invention an apparatus and method is provided that can be utilized to quickly and accurately position an interventional device in relation to a lesion of interest by first identifying the location of the lesion in two dimensions using nuclear emission imaging and then by determining the third dimension using ultrasound imaging applied precisely at the two-dimensional location predefined by nuclear emission imaging. In this way, the lesion of interest may be determined by ultrasound even though it may not be otherwise distinguishable from surrounding abnormalities. Once the lesion has been identified, the task of interventional device localization is simplified using the recognized advantages of real-time structure visualization afforded by ultrasound imaging.

### BRIEF DESCRIPTION OF THE DRAWINGS

FIG. 1 illustrates a cross sectional side view of one embodiment of a nuclear-emission imager with ultrasound correlation for body part imaging for interventions according of the present invention.

FIG. 2 illustrates a perspective view of the nuclear-emission imager with ultrasound correlation for body part imaging of FIG. 1.

### DETAILED DESCRIPTION OF THE INVENTION

Nuclear-emission imaging is sometimes used to identify abnormal cellular function(s) (e.g. glucose metabolism as with FDG-PET, mitochondrial proliferation as with sestamibi-scintigraphy) of a region of tissue (lesion) that may be associated with tumor growth (neoplasia). However, it is difficult to use nuclear-emission imaging alone to guide interventions due to the long image acquisition periods (e.g. five minutes), unacceptable nuclear-emission image spatial resolution and the transparency of most interventional devices to emission imaging.

Ultrasound imaging may be utilized to identify very subtle abnormalities in tissue density and structure that commonly result from neoplasia. However, abnormalities produced by cancer are difficult to distinguish from abnormalities produced by benign processes using ultrasound, and ultrasound alone is considered to have unacceptable specificity for early breast cancer detection. Notwithstanding, it is commonly understood that most suspect lesions found by imaging methods that are used for early breast cancer detection such as x-ray mammography, MRI or nuclear emission imaging have developed some degree of associated anatomical abnormality that can be found by carefully surveying the region of the suspect lesion with ultrasound imaging, referred to as ‘second pass ultrasound’. The difficulty becomes confining the ultrasound survey to the region of the suspect lesion so as to preclude or reduce the possibility of another lesion being mistaken for the suspect lesion. An example of a confined region may be along a line specified as normal to a plane where the line is defined by two dimensions through the plane. In this regard, the specificity of ultrasound for identifying a suspect lesion can be improved by confining its survey to the region of the suspect lesion. An example of an unconfined region may be a specified breast quadrant.

Interventional guidance is highly simplified by ultrasound imaging once the suspect lesion in question is identified because the trajectory of the interventional device can be adjusted in real time during insertion and proper tissue harvesting or marking may easily be verified with real-time visualization of both the lesion and the interventional device. The present invention may enable lesions identified on nuclear-emission imaging to be identified with high specificity using ultrasound imaging. An apparatus and method for enabling the nuclear-emission and ultrasound correlated intervention will be further described below.

The present invention provides an apparatus and method for localizing a lesion that has been identified on a nuclear-emission image using correlated nuclear and ultrasound imaging. The method combines the steps of stabilizing the tissue in relation to the nuclear imager, obtaining a nuclear-emission image, determining the location of the lesion in two dimensions relative to the nuclear imager (e.g. x-y coordinates that are normal to the plane of the detector), providing interventional access, positioning an ultrasound transducer facing the lesion using the nuclear-emission identified lesion coordinates, identifying the likely lesion using ultrasound, and using the ultrasound image to guide and verify the intervention. Some embodiments may further confirm the lesion was altered using nuclear-emission imaging.

One embodiment of the apparatus according to the present invention may include a device for immobilizing the body part with respect to the space-coordinates of the nuclear-emission imager. Some embodiments may also include a device for shielding the field of view of the nuclear-emission imager from stray radiation during image acquisition that also provides intermittent access for interventions while maintaining immobilization. Some embodiment may also use a rigid localization fixture for positioning an ultrasound transducer normal to a plane at the two-dimensional coordinates determined by nuclear-emission imaging.

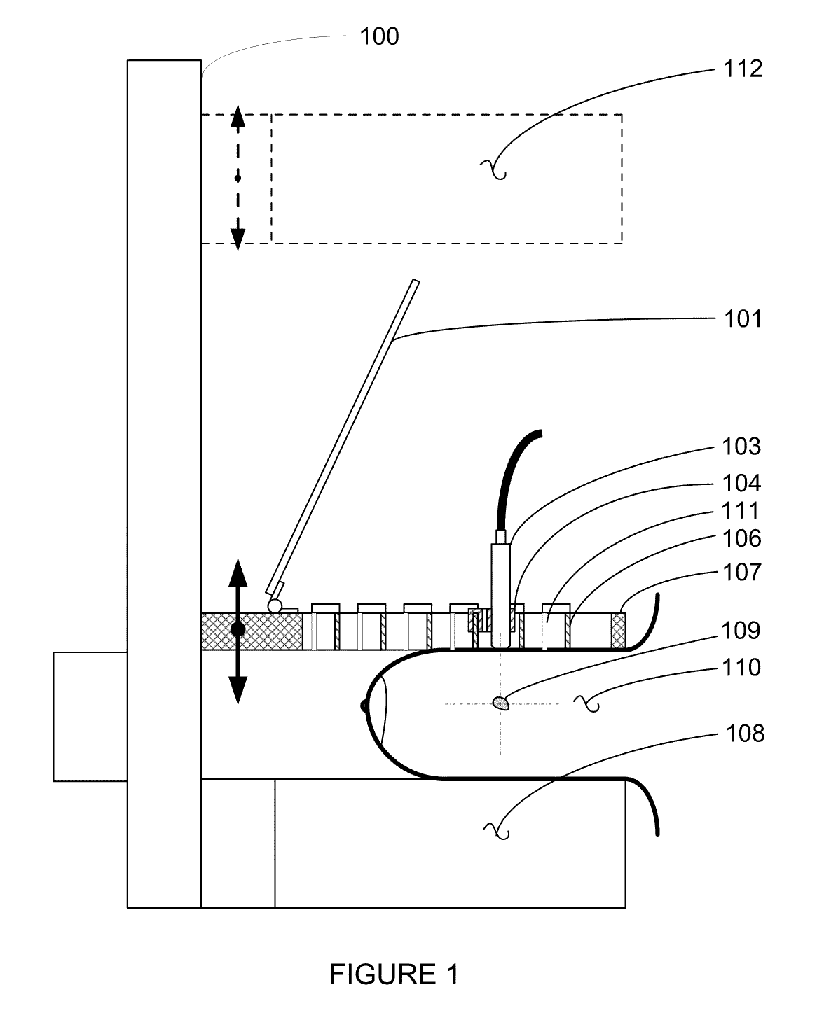

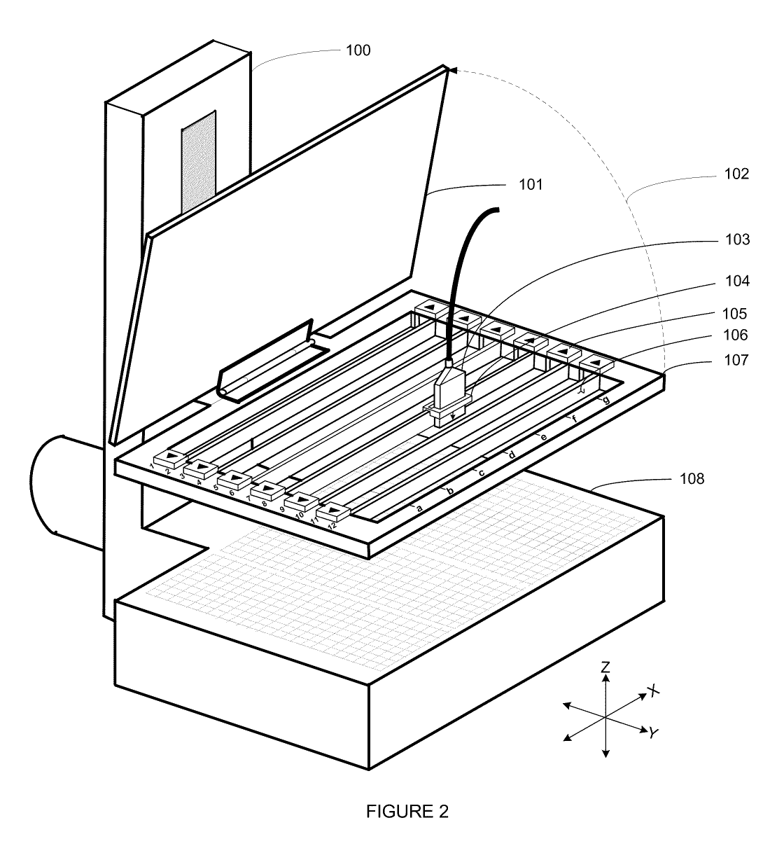

As shown in FIG. 1, a body part 110 has been immobilized by compression and readied for an intervention on one embodiment of a nuclear-emission imager with ultrasound correlation apparatus 100. A lesion 109 has been identified in at least the x and y-axis locations shown as shown in FIG. 2 using a single gamma-ray detector 108. As shown in FIG. 1, some embodiments may use a second gamma ray detector 112, which may be needed for coincidence imaging or dual-detector imaging, displaced opposing the single gamma-ray detector 108 to provide access to body part 110. An ultrasound transducer 103 may be precisely positioned and stabilized to focus on and capture images in the region of lesion 109 at said x and y locations (e.g. location D-E;8 as shown in FIG. 2) by the adjustable frame 104. The ultrasound transducer 103 may be repositioned without sliding across the surface of the body part 110 (e.g. skin) to prevent unwanted body part movement that could disturb the location of the lesion. To reposition the ultrasound transducer 103, the operator may release the ultrasound transducer 103 from a beam 106 of the adjustable frame 104 and relocate the ultrasound transducer 103 to a more desired location. Embodiments that include a shielding panel 101 may be used with a single gamma ray detector 108 and can be moved to a raised position 102 to provide access to the body part 110 for positioning the ultrasound transducer 103 and performing the intervention.

Once the operator identifies the lesion 109 using images produced by the ultrasound transducer 103, an intervention can be initiated. Various interventional access methods may be provided by the apparatus of the present invention including between the compression frame 107 and the nuclear emission imager 108 and aside the beams 106 of the compression frame 107. For example, an interventional device can be placed in the same position on the beam 106 where the ultrasound transducer 103 was placed to locate the lesion 109. In the same manner in which the ultrasound transducer 103 may be relocated, an intervention device may also be relocated to adjacent slots (e.g. 111) to provide full access to all locations within the compression frame 107. To relocate a single compression beam 106, spring-loaded tabs 105 may be slid outward as depicted by the arrows on the compression frame 107. The spring-loaded tabs 105 may release the compression beam 106 so it can be removed up and out of its slot, then installed back down into an adjacent slot, while immobilization of the body part 110 and the lesion 109 is maintained by the neighboring beams of the compression frame 107.

It should be understood, of course, that the foregoing relates to exemplary embodiments of the invention and that modifications may be made without departing from the spirit and scope of the invention as set forth in the following claims. Accordingly the embodiments of the invention described herein are merely illustrative of the application of the principles of the invention. For example, the use of MRI or optical imaging instead of nuclear-emission imaging, or the use of various types nuclear emission imaging including but not limited to PET, dual-head scintigraphy and Compton imaging may also be used to achieve the same. Reference herein to details of the illustrated embodiments is not intended to limit the scope of the claims, which themselves recite those features regarded as essential to the invention.

What is claimed is:

1. An apparatus comprising:
a nuclear emission imaging device;
a compression frame for immobilizing a body part containing a lesion, wherein the compression frame is adjustable to move away from and/or toward the nuclear emission imager in order to secure the body part between the nuclear emission imager and the compression frame, and wherein the compression frame comprises markings to convey a planar (x,y) position of the lesion as determined by the nuclear emission imager;
an adjustable frame, wherein the adjustable frame is coupled to the compression frame;
a plurality of slots on the adjustable frame;
a plurality of beams that may slide into a pair of slots, wherein a first beam can be repositioned to a different pair of slots on the adjustable frame; and
an ultrasound transducer, wherein the ultrasound transducer is positioned on the beam to allow the ultrasound transducer to be placed on the body part and aimed at the lesion without altering the position of the body part.

2. The apparatus of claim 1 further comprising a radiation shield coupled to the compression frame, wherein the radiation shield is adjustable to allow access to the body part after nuclear emission imaging of the body part.

3. The apparatus of claim 1 further comprising a second nuclear emission imaging device displaced in opposition to the compression frame.

4. An apparatus comprising:
a nuclear emission imaging device;
an adjustable compression frame, wherein the adjustable compression frame is adjustable to move away from and/or toward the nuclear emission imaging device in order to immobilize a body part between the nuclear emission imaging device and the adjustable compression frame; and
an ultrasound transducer, wherein the ultrasound transducer is coupled to the compression frame to allow contact with the body part without altering the position of the body part.

5. The apparatus of claim 4 further comprising:
a plurality of slots on the adjustable compression frame; and
at least one beam positioned on a pair of slots, wherein the beam is for accommodating the ultrasound transducer, and wherein the beam is able to be repositioned to another pair of slots.

6. The apparatus of claim 5, wherein the beam is further for accommodating an interventional device in order to position the intervention device to target a lesion through tissue of the body part.

7. The apparatus of claim 5 further comprising a plurality of spring-loaded tabs above each slot to allow the beams to be positioned securely within a pair of slots.

8. The apparatus of claim 4 further comprising a radiation shield coupled to the compression frame, wherein the radiation shield is adjustable to allow access to the body part after nuclear emission imaging of the body part.

9. The apparatus of claim 4 further comprising a second nuclear emission imaging device displaced in opposition to the compression frame.

10. The apparatus of claim 4 further comprising a magnetic resonance imaging device for identifying a lesion in the body part.

11. The apparatus of claim 4 further comprising markings on the adjustable frame to convey the planar (x,y) position of a lesion in the body part as determined by the nuclear emission imaging device.

12. A method for performing an intervention of a lesion within a body part, the method comprising:
immobilizing the body part between a nuclear emission imaging device and a compression frame;
identifying the lesion in the body part using the nuclear emission imaging device;
positioning an ultrasound transducer on the body part where the lesion is identified;
identifying a precise location of the lesion using the ultrasound transducer; and
performing an intervention on the lesion, wherein guidance of the intervention is provided by using an ultrasound image provided by the ultrasound transducer.

13. The method of claim 12 further comprising:
positioning a radiation shield above the nuclear emission imaging device to prevent unwanted energy from entering the nuclear emission imaging device.

14. The method of claim 12 further comprising:
re-imaging the body part using the nuclear emissions imaging device to verify whether the lesion is altered by the intervention.

15. The method of claim 12 further comprising:
using a second nuclear emission imaging device to identify the lesion in the body part for coincidence imaging.

16. The method of claim 12 further comprising:
replacing the ultrasound transducer with an interventional device to target the lesion through tissue of the body part.
